(19) United States
(12) Patent Application Publication
(10) Pub. No.: US 2025/0364136 A1
(43) Pub. Date: Nov. 27, 2025

(54) LUNG VOLUME ESTIMATION METHOD AND APPARATUS

(71) Applicant: MEDICALIP CO., LTD., Gangwon-do (KR)

(72) Inventors: Sang Joon Park, Seoul (KR); Soon Ho Yoon, Seoul (KR); Hyungjin Kim, Seoul (KR); Joseph Nathanael Witanto, Seoul (KR); Jong Min Kim, Gyeonggi-do (KR)

(73) Assignee: MEDICALIP CO., LTD., Gangwon-do (KR)

(21) Appl. No.: 18/674,451

(22) Filed: May 24, 2024

(51) Int. Cl. G16H 50/20; G06T 7/00; G06T 7/11; G16H 10/60; G16H 30/40

(52) U.S. Cl. CPC G16H 50/20; G06T 7/0012; G06T 7/11; G16H 10/60; G16H 30/40; G06T 2207/10004; G06T 2207/10116; G06T 2207/20081; G06T 2207/20084; G06T 2207/30061

(57) ABSTRACT

Provided are a lung volume estimation method and an apparatus therefor. The lung volume estimation apparatus inputs two-dimensional medical image and clinical information of a patient into an artificial intelligence model to determine the patient's lung volume. The artificial intelligence model may include a first neural network that generates an encoded image for the two-dimensional medical image and a second neural network that determines a lung volume from data obtained by concatenating an output value of the first neural network with the clinical information.

### FIELD

The disclosure relates to a lung volume estimation method and an apparatus therefor, and more particularly, to a lung volume estimation method of estimating a lung volume from medical images, and an apparatus therefor.

### BACKGROUND

In the conventional art, lung volume is measured using a spirometer based on the amount of air a patient has inhaled and exhaled. However, when lungs become stiff due to disorders such as pulmonary fibrosis, the elasticity of the lungs decreases, making it difficult to exhale, and more air is trapped in the lungs, which reduces the accuracy of lung volume using conventional spirometers.

Lung volume may be obtained by performing a process of separating a lung region from a computed tomography (CT) image of a patient's chest. However, in order to obtain the lung volume, a costly and time-consuming CT scan should be preceded, and a complex process of separating and extracting lung regions from 3D medical images is also required.

### SUMMARY

Provided are a lung volume estimation method and an apparatus therefor, in which a patient's lung volume is easily determined from a two-dimensional medical image such as an X-ray image.

Additional aspects will be set forth in part in the description which follows and, in part, will be apparent from the description, or may be learned by practice of the presented embodiments of the disclosure.

According to an aspect of the disclosure, a lung volume estimation method, includes receiving a two-dimensional medical image of a patient, receiving clinical information of the patient, and determining a lung volume of the patient by inputting the two-dimensional medical image and the clinical information into an artificial intelligence model.

According to another aspect of the disclosure, a lung volume estimation apparatus, includes an image input unit configured to receive a two-dimensional medical image of a patient, a clinical information input unit configured to receive clinical information of the patient, and a volume determination unit configured to determine a lung volume of the patient through an artificial intelligence model configured to output a lung volume by receiving the two-dimensional medical image and the clinical information as inputs.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram showing an example of a lung volume estimation apparatus according to an embodiment;

FIG. 2 is a flowchart illustrating an example of a lung volume estimation method according to an embodiment;

FIG. 3 is a diagram showing a structure of an example of an artificial intelligence model according to an embodiment;

FIGS. 4 and 5 are diagrams illustrating an example of a region segmentation model according to an embodiment;

FIGS. 6 to 8 are diagrams showing structures of various embodiments of an artificial intelligence model according to an embodiment;

FIG. 9 is a diagram showing an example of training data of an artificial intelligence model according to an embodiment; and

FIG. 10 is a diagram illustrating a configuration of an example of a lung volume estimation apparatus according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, a lung volume estimation method and an apparatus therefor according to an embodiment will be described in detail with reference to the accompanying drawings.

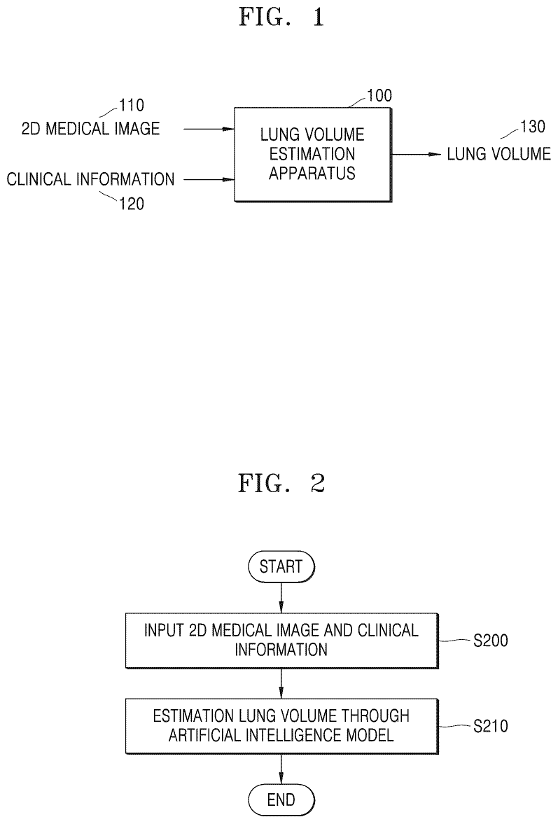

FIG. 1 is a diagram showing an example of a lung volume estimation apparatus according to an embodiment.

Referring to FIG. 1, a lung volume estimation apparatus 100 determines and outputs a lung volume 130 when receiving a two-dimensional (2D) medical image 110 and clinical information 120. In an embodiment, the lung volume estimation apparatus 100 may receive a 2D medical image 110 and clinical information 120 from a Picture Archiving and Communication System (PACS) and an Electronic Medical Record (EMR) system.

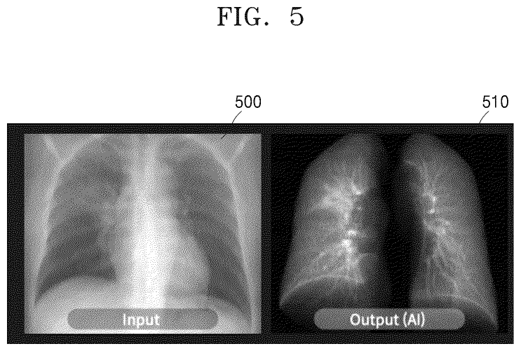

The 2D medical image 110 may be a chest X-ray image. Alternatively, the 2D medical image 110 may be a segmented image obtained by dividing only a lung region from a chest X-ray image. An example of a method of generating a segmented image of a lung region from an X-ray image is shown in FIGS. 4 and 5.

Since the lung volume 130 has a significant correlation with the age and/or gender of the patient, the clinical information 120 may include age and/or gender. In another embodiment, the clinical information 120 may further include quantitative data such as a size of a lung region along with age and gender. In addition, various information having a correlation with the lung volume may be included in the clinical information 120, and the clinical information 120 is not limited to the present embodiment. However, hereinafter, a case in which age and/or gender are included as an example of the clinical information 120 will be mainly described.

FIG. 2 is a flowchart illustrating an example of a lung volume estimation method according to an embodiment.

Referring to FIG. 2, the lung volume estimation apparatus 100 (hereinafter, referred to as an “apparatus”) receives a 2D medical image and clinical information (S200). The 2D medical image may be a chest X-ray image or a segmented image obtained by separating a lung region from the chest X-ray image. The clinical information may include age, gender, and the like.

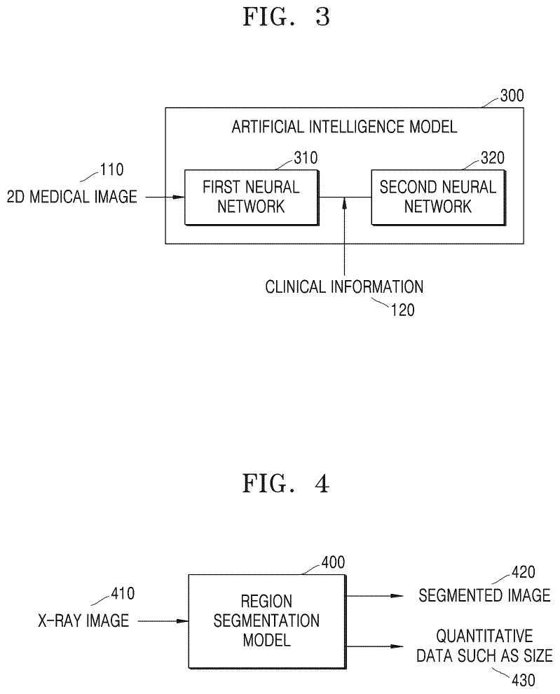

The apparatus 100 inputs the 2D medical image and the clinical information into an artificial intelligence model to estimate a lung volume (S210). For example, an artificial intelligence model may be implemented as an artificial neural network that predicts and outputs a lung volume when receiving a 2D medical image and clinical information. 2D medical images and clinical information have different data types. The present embodiment proposes an artificial intelligence model including a first neural network and a second neural network to accurately predict a lung volume based on two different types of data. An example of the structure of an artificial intelligence model according to an embodiment is shown in FIG. 3.

FIG. 3 is a diagram showing a structure of an example of an artificial intelligence model according to an embodiment.

Referring to FIG. 3, an artificial intelligence model 300 includes a first neural network 310 and a second neural network 320. The first neural network 310 and the second neural network 320 may be the same type of artificial neural network or different types of artificial neural networks. For example, the first neural network 310 may be a U-Net, and the second neural network 320 may be a convolutional neural network (CNN). In addition, the first neural network 310 and the second neural network 320 may be implemented with various types of conventional artificial neural networks, and are not limited to a specific type. However, hereinafter, for convenience of explanation, a case in which the first neural network 310 is a U-Net will be mainly described.

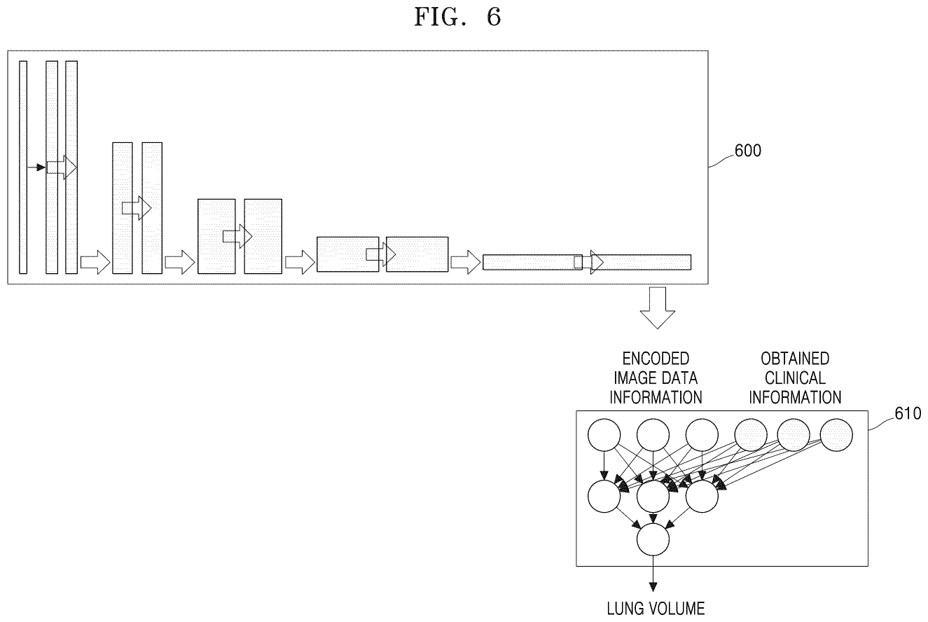

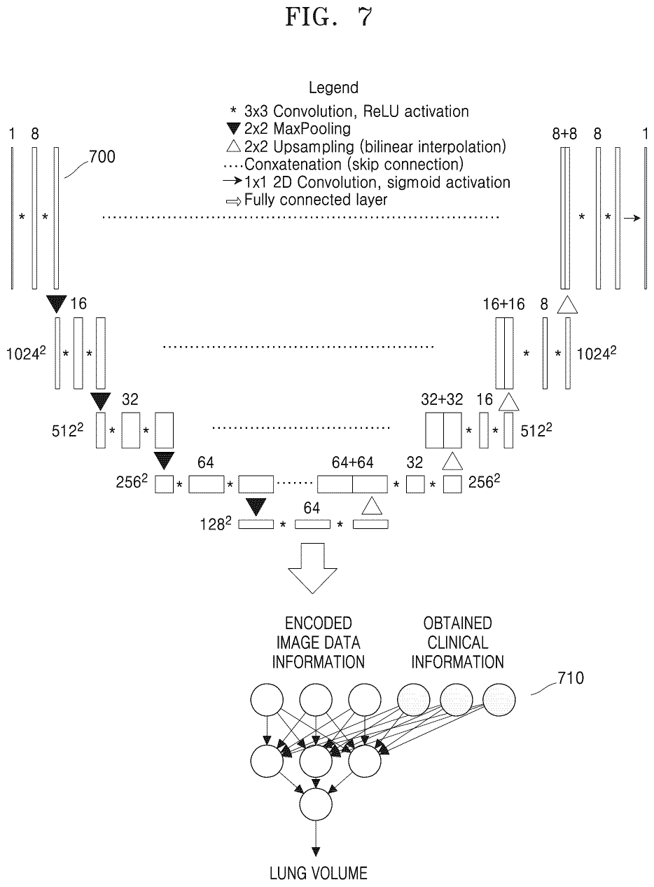

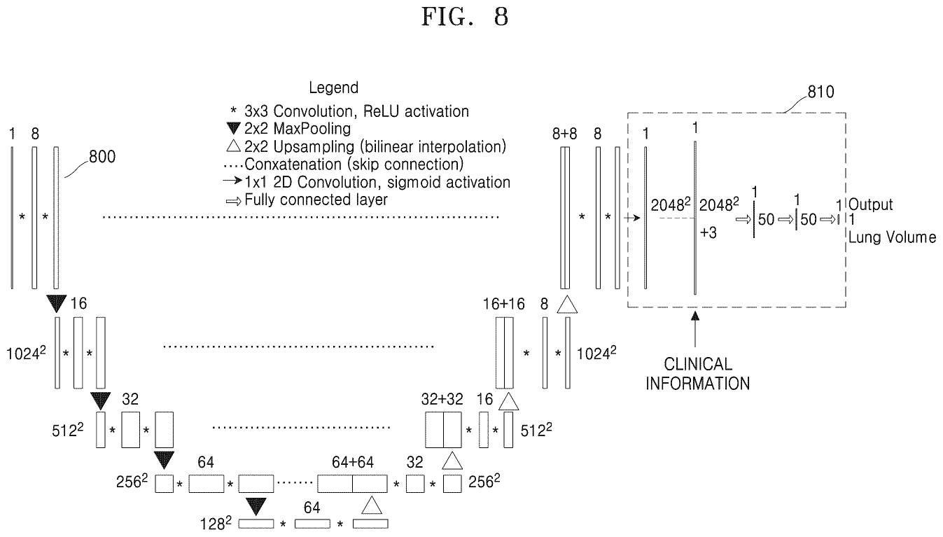

The first neural network 310 receives a 2D medical image 110. The first neural network 310 may use various conventional artificial neural networks such as U-Net as they are or may be modified based on the same. As an example, the first neural network 310 may be a model that outputs an encoded image (or feature map) obtained by encoding (or downsampling) the 2D medical image 110 (see FIG. 6). In another embodiment, the first neural network 310 may be a model that outputs a reconstructed image by performing an encoding process (i.e., downsampling) and a decoding process (i.e., upsampling) when receiving a 2D medical image (see FIGS. 7 and 8).

The second neural network 320 is a model that outputs a lung volume when receiving data obtained by concatenating the output value of the first neural network 310 with the clinical information 120. The output value of the first neural network 310 may be feature information output from a specific layer among a plurality of layers constituting the first neural network 310. Examples of various concatenation structures of the first neural network 310 and the second neural network 320 are shown in FIGS. 6 to 8.

FIGS. 4 and 5 are diagrams illustrating an example of a region segmentation model according to an embodiment.

Referring to FIG. 4, when receiving an X-ray image 410, the region segmentation model 400 is an artificial intelligence model that generates a segmentation image 420 of a lung region from the X-ray image 410. An example of the segmented image 510 obtained by separating a lung region from the chest X-ray image 500 is shown in FIG. 5. In the X-ray image 500, several tissues (lungs, bones, muscles, organs, etc.) are overlapped and displayed on one plane, but in the segmented image 510, only lung tissues are displayed. When the segmented image 510 of the lung region is used as a 2D medical image, the accuracy of lung volume measurement may be improved.

The region segmentation model 400 may be implemented with various artificial neural networks such as CNNs. The region segmentation model 400 may be trained and generated through a supervised learning method. For example, the region segmentation model 400 may be trained using training data including an X-ray image 410 and a 2D image (ground truth) of a lung region as a dataset. That is, when receiving the X-ray image 410 of the training data, the region segmentation model 400 outputs the segmented image 420 and may be trained to reduce the difference between the segmented image 420 and the 2D image (ground truth) of the lung region of the training data. Since the method of training the region segmentation model 400 by the supervised learning method using the training data itself is a known technique, an additional description thereof is omitted.

In an embodiment, the training data may be generated based on a CT image. For example, 3D lung regions are segmented from chest CT images using various conventional segmentation algorithms. In addition, a 2D image generated by projecting a chest CT image onto a 2D plane may be used as an X-ray image of training data, and a 2D lung image generated by projecting a three-dimensional (3D) lung region onto a 2D plane may be used as a ground truth. In addition, training data for the region segmentation model 400 may be generated in various ways and is not limited to the present embodiment.

The apparatus 100 may obtain quantitative data 430 such as a size of a lung region from the segmented image 420 obtained through the region segmentation model 400. The apparatus 100 may use a size of a lung region as clinical information.

FIG. 6 is a diagram showing a structure of a first example of an artificial intelligence model according to an embodiment.

Referring to FIG. 6, an artificial intelligence model includes a first neural network 600 that performs an encoding process (e.g., downsampling) and a second neural network 610 that predicts a lung volume.

When the first neural network 600 receives a 2D medical image, the first neural network 600 outputs an encoded image obtained by encoding the 2D medical image. The first neural network 600 may be configured to include an encoder portion while omitting a decoder portion from various types of artificial neural networks including an encoder and a decoder. For example, the first neural network 600 may include layers (i.e., encoders) that perform an encoding process in a U-Net.

When the second neural network 610 receives the output value (i.e., encoded image) from the first neural network 600 and the clinical information, the second neural network 610 outputs a lung volume. The second neural network 610 may be implemented as various types of artificial neural networks such as CNNs.

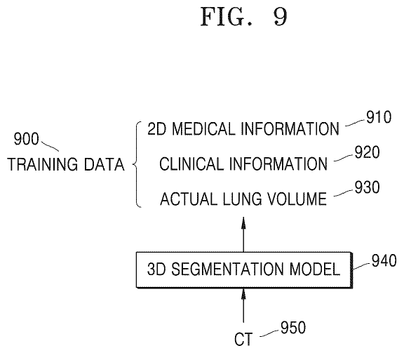

The apparatus 100 may train an entire artificial intelligence model to which the first neural network 600 and the second neural network 610 are connected. For example, an artificial intelligence model may be trained and generated through a regression estimation method. In another embodiment, the apparatus 100 may train each of the first neural network 600 and the second neural network 610 or may train only the second neural network 610. A method of training an artificial intelligence model using training data will be described again with respect to FIG. 9.

FIG. 7 is a diagram showing a structure of a second example of an artificial intelligence model according to an embodiment.

Referring to FIG. 7, an artificial intelligence model includes a first neural network 700 that performs an encoding process and a decoding process, and a second neural network 710 that predicts a lung volume.

The first neural network 700 includes an encoder that encodes a 2D medical image when receiving 2D medical image, and a decoder that decodes the encoded data to generate a reconstructed image. For example, the first neural network 700 may be implemented as a U-Net. The first neural network 700 may be trained to reduce a difference between a 2D medical image as input data and a reconstructed image as output data. For example, the first neural network 700 may be trained with a self-supervised learning method.

When receiving data obtained by concatenating feature information (e.g., encoded image) output from the encoder of the first neural network 700 with the clinical information, the second neural network 710 outputs a lung volume. The second neural network may be implemented as the same artificial neural network as the second neural network 610 illustrated in FIG. 6 or may be implemented as different artificial neural networks.

The first neural network 700 and the second neural network 710 of the present embodiment may be generated through separate learning processes, respectively. Training methods of the first neural network 700 and the second neural network 710 will be described again with respect to FIG. 9.

FIG. 8 is a diagram showing a structure of a third example of an artificial intelligence model according to an embodiment.

Referring to FIG. 8, an artificial intelligence model includes a first neural network 800 that performs an encoding process and a decoding process, and a second neural network 810 that predicts a lung volume.

The first neural network 800 includes an encoder that encodes a 2D medical image when receiving 2D medical image, and a decoder that decodes the encoded data to reconstruct an image again. For example, the first neural network 800 may be implemented as a U-Net.

The second neural network 810 is located at an output end of the first neural network 800. When receiving data obtained by concatenating a reconstructed image output from the first neural network 800 with clinical information, the second neural network 810 outputs a lung volume.

The apparatus 100 may train an entire artificial intelligence model to which the first neural network 800 and the second neural network 810 are connected. In another embodiment, the apparatus 100 may train each of the first neural network 800 and the second neural network 810 or may train only the second neural network 810. A method of training an artificial intelligence model using training data will be described again with respect to FIG. 9.

FIG. 9 is a diagram showing an example of training data of an artificial intelligence model according to an embodiment.

Referring to FIG. 9, training data 900 of an artificial intelligence model includes a dataset of a 2D medical image 910, clinical information 920, and an actual lung volume 930. The 2D medical image 910 may be a chest X-ray image or a segmented image described with reference to FIGS. 4 and 5. The clinical information 920 may include the age and gender of the patient. In another embodiment, the clinical information 920 may further include an area of a lung region obtained from the segmented image of FIG. 4. The actual lung volume 930 may be determined through a 3D segmentation model 940 or the like that segments a 3D lung region from a CT image 950. The 3D segmentation model 940 may be various conventional models implemented as artificial intelligence models. In addition, the actual lung volume of training data may be determined through various conventional methods.

The apparatus 100 may train various artificial intelligence models of FIGS. 6 to 9 using the training data 900.

First, a learning method of the artificial intelligence model of FIG. 6 is described below.

The apparatus 100 inputs the 2D medical image 910 of the training data 900 to the first neural network 600. The apparatus 100 inputs data obtained by concatenating the encoded image output from the first neural network 600 with the clinical information 920 of the training data 900 to the second neural network 610 to predict a lung volume. Hereinafter, the lung volume predicted and output by the second neural network 610 is referred to as a “predicted lung volume.” The apparatus 100 trains the first neural network 600 and the second neural network 610 together based on a loss function indicating a difference between the predicted lung volume and the actual lung volume 930 of the training data 900. The loss function is a loss function for the first neural network 600 and the second neural network 610 as a whole. The apparatus 100 performs a training process to adjust values of parameters of the first neural network 600 and the second neural network 610 so that the value of the loss function decreases.

Next, a learning method of the artificial intelligence model of FIG. 7 is described below.

The apparatus 100 inputs the 2D medical image 910 of the training data 900 to a first neural network 700. The first neural network 700 outputs a reconstructed image through encoding and decoding processes. The first neural network 700 performs a first training process of adjusting the values of the parameters of the first neural network 700 so that the value of the loss function indicating the difference between the 2D medical image 910 of the training data 900 and the reconstructed image decreases. That is, the first neural network 700 may be generated by self-supervised learning.

When the first training process is completed, the apparatus 100 inputs the 2D medical image 910 of the training data 900 to the training-completed first neural network 700. The apparatus 100 inputs data obtained by concatenating the feature information (i.e., encoded image) of the encoder of the first neural network 700 with the clinical information 920 of the training data 900 to the second neural network 710 to determine the predicted lung volume. The apparatus 100 performs a second training process of adjusting values of the parameters of the second neural network 710 to reduce the value of the loss function indicating the difference between the predicted lung volume and the actual lung volume 930 of the training data 900. Since the first neural network 700 has been trained in advance, the learning state of the first neural network 700 is fixed. That is, in the second training process, the second neural network 710 excluding the first neural network 700 is trained.

Next, a learning method of the artificial intelligence model of FIG. 8 is described below.

The apparatus 100 inputs the 2D medical image 910 of the training data 900 to the first neural network 800 to obtain a reconstructed image. The apparatus 100 inputs, to the second neural network 810, data obtained by concatenating the reconstructed image output from the first neural network 800 with the clinical information to determine the predicted lung volume. The apparatus 100 performs a training process of adjusting values of the parameters of the first neural network 800 and the second neural network 810 to reduce the value of the loss function indicating the difference between the predicted lung volume and the actual lung volume 930 of the training data 900. The artificial intelligence model of FIG. 7 trains the first neural network 700 and the second neural network 710, respectively, whereas the artificial intelligence model of FIG. 8 trains the first neural network 800 and the second neural network 810 together at a time.

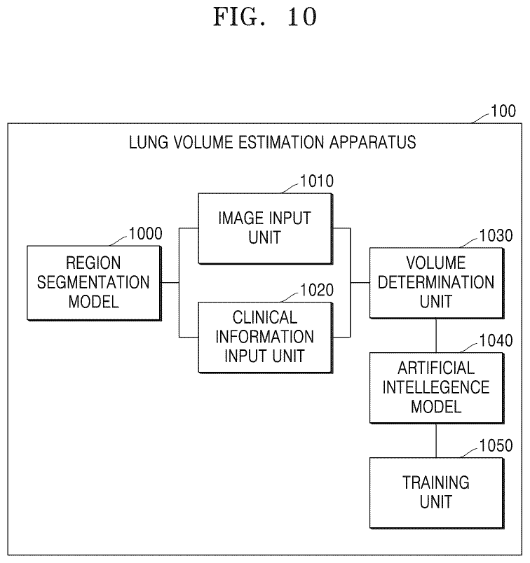

FIG. 10 is a diagram illustrating a configuration of an example of a lung volume estimation apparatus according to an embodiment.

Referring to FIG. 10, the lung volume estimation apparatus 100 includes a region segmentation model 1000, an image input unit 1010, a clinical information input unit 1020, a volume determination unit 1030, an artificial intelligence model 1040, and a training unit 1050. Depending upon an embodiment, the region segmentation model 1000 may be omitted. In another embodiment, if the artificial intelligence model 1040 has been trained in advance, the training unit 1050 may be omitted. In another embodiment, the lung volume estimation apparatus 100 may be implemented as a computing device including a memory, a processor, and an input/output device. In this case, each component may be implemented in software and then performed by a processor after being loaded on a memory.

The image input unit 1010 receives a 2D medical image of a patient. In an embodiment, the image input unit 1010 may receive a chest X-ray image as a 2D medical image. In another embodiment, the image input unit 1010 may receive a segmented image including a lung region segmented from the X-ray image as a 2D medical image through the region segmentation model 1000.

The clinical information input unit 1020 receives clinical information of a patient. In an embodiment, the clinical information input unit 1020 may receive age and gender of a patient as clinical information. In another embodiment, the clinical information input unit 1020 may receive quantitative data such as the size of the lung region determined through the region segmentation model 1000 and the age and gender of the patient as clinical information.

The volume determination unit 1030 determines the patient's lung volume through the artificial intelligence model 1040 that outputs the lung volume by receiving the 2D medical image and clinical information as inputs.

A first example of the artificial intelligence model 1040 may include a first neural network configured to encode an image and a second neural network configured to predict a lung volume. In this case, the volume determination unit 1030 may generate an encoded image for the 2D medical image through the first neural network, and input, to the second neural network, data obtained by concatenating the encoded image with clinical information to determine the lung volume. The first example of the artificial intelligence model is illustrated in FIG. 6.

A second example of the artificial intelligence model may include a first neural network configured to encode and reconstruct an image through an encoder and a decoder, and a second neural network configured to predict a lung volume. In this case, the volume determination unit 1030 may determine the lung volume by inputting, to the second neural network, data obtained by concatenating the feature information of the encoder on the 2D medical image with clinical information. The second example of the artificial intelligence model is illustrated in FIG. 7.

A third example of the artificial intelligence model may include a first neural network configured to encode and reconstruct an image through an encoder and a decoder, and a second neural network configured to predict a lung volume. In this case, the volume determination unit 1030 may obtain a reconstructed image for the 2D medical image through the first neural network, and input, to the second neural network, data obtained by concatenating the reconstructed image with clinical information to determine the lung volume. The third example of the artificial intelligence model is illustrated in FIG. 8.

The training unit 1050 trains an artificial intelligence model including the first neural network and the second neural network using the training data through a supervised learning method. In the case of the first artificial intelligence model of FIG. 6, the training unit 1050 may input a 2D medical image of training data to the first neural network that performs image encoding, input, to the second neural network, data obtained by concatenating the output value of the first neural network with clinical information of training data to predict a lung volume, and train the first neural network and the second neural network together to reduce the difference between the predicted lung volume and the actual lung volume of the training data.

In the case of the second artificial intelligence model of FIG. 7, the training unit 1050 may include a first training unit and a second training unit. The first training unit may train the first neural network by a self-supervised learning method by inputting a 2D medical image of training data to the first neural network that performs image encoding and image decoding. The second training unit may determine feature information on the 2D medical image of the training data through the training-completed first neural network, predict a lung volume by inputting, to the second neural network, data obtained by concatenating the feature information with the clinical information of the training data, and train the second neural network to reduce the difference between the predicted lung volume and the actual lung volume of the training data.

In the case of the third artificial intelligence model of FIG. 8, the training unit 1050 may generate a reconstructed image by inputting a 2D medical image of training data to the first neural network that performs image encoding and image decoding, predict a lung volume by inputting, to the second neural network, data obtained by concatenating the reconstructed image with clinical information of the training data, and train the first neural network and the second neural network together to reduce the difference between the predicted lung volume and the actual lung volume of the training data.

The lung volume estimation method according to the disclosure may also be implemented as computer-readable program code on a computer-readable recording medium. The computer-readable recording medium includes all types of recording devices in which data readable by a computer system is stored. Examples of computer-readable recording media include ROM, RAM, CD-ROM, magnetic tape, floppy disk, optical data storage device, and the like. In addition, computer-readable recording media are distributed in a network-connected computer system so that computer-readable code may be stored and executed in a distributed manner.

According to embodiments, it is possible to determine the lung volume from a 2D medical image such as an X-ray image or the like. In addition, the accuracy of lung volume measurement may be improved by using clinical information along with 2D medical images.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

We claim:

1. A lung volume estimation method, comprising:
receiving a two-dimensional (2D) medical image of a patient;
receiving clinical information of the patient; and
determining a lung volume of the patient by inputting the 2D medical image and the clinical information into an artificial intelligence model.

2. The method of claim 1, wherein the 2D medical image comprises a chest X-ray image.

3. The method of claim 1, wherein the clinical information comprises an age or gender of the patient.

4. The method of claim 1, wherein the 2D medical image comprises a segmented image obtained by separating a lung region from an X-ray image.

5. The method of claim 4, further comprising generating the segmented image of the lung region from the 2D medical image using a region segmentation model trained using training data including an X-ray image and a segmented image of a lung region.

6. The method of claim 4, wherein the clinical information comprises an area of the lung region determined based on the segmented image.

7. The method of claim 1, wherein the determining of the lung volume comprises:
generating an encoded image for the 2D medical image through a first neural network; and
predicting a lung volume by inputting, into a second neural network, data obtained by concatenating the encoded image with the clinical information.

8. The method of claim 7, further comprising training the first neural network and the second neural network through a supervised learning method by using training data including 2D medical images, clinical information, and an actual lung volume.

9. The method of claim 1, wherein the determining of the lung volume comprises:
inputting the 2D medical image into a first neural network including an encoder and a decoder; and
predicting a lung volume by inputting, into a second neural network, data obtained by concatenating feature information output from the encoder with the clinical information.

10. The method of claim 9, further comprising
training the artificial intelligence model including the first neural network and the second neural network, wherein
the training of the first neural network comprises:
inputting the 2D medical image into the first neural network; and
training the first neural network to reduce a difference between a reconstructed image output by the first neural network and the 2D medical image, and
the training of the second neural network comprises:
inputting the 2D medical image into the training-completed first neural network; and
training the second neural network to reduce a difference between the predicted lung volume and an actual lung volume, the predicted lung volume being obtained by inputting, into the second neural network, the data obtained by concatenating the feature information output from the encoder of the first neural network with the clinical information.

11. The method of claim 1, wherein the determining of the lung volume comprises:
generating a reconstructed image by inputting the 2D medical image into a first neural network including an encoder and a decoder; and
predicting a lung volume by inputting, into a second neural network, data obtained by concatenating the reconstructed image with the clinical information.

12. The method of claim 11, further comprising training the first neural network and the second neural network through a supervised learning method by using training data including 2D medical images, clinical information, and an actual lung volume.

13. A lung volume estimation apparatus, comprising:
an image input unit configured to receive a two-dimensional (2D) medical image of a patient;
a clinical information input unit configured to receive clinical information of the patient; and
a volume determination unit configured to determine a lung volume of the patient through an artificial intelligence model configured to output a lung volume by receiving the 2D medical image and the clinical information as inputs.

14. The apparatus of claim 13, wherein the artificial intelligence model comprises:
a first neural network configured to encode an image; and
a second neural network configured to predict the lung volume, wherein
the volume determination unit is further configured to generate an encoded image for the 2D medical image through the first neural network, and input data obtained by concatenating the encoded image with the clinical information into the second neural network to determine the lung volume.

15. The apparatus of claim 13, wherein the artificial intelligence model comprises:
a first neural network configured to encode and decode an image through an encoder and a decoder; and
a second neural network configured to predict the lung volume, wherein
the volume determination unit is further configured to input data obtained by concatenating feature information on the 2D medical image, determined through the encoder, with the clinical information into the second neural network to determine the lung volume.

16. The apparatus of claim 13, wherein the artificial intelligence model comprises:
a first neural network configured to encode and decode an image through an encoder and a decoder; and
a second neural network configured to predict the lung volume, wherein
the volume determination unit is further configured to obtain a reconstructed image for the 2D medical image through the first neural network, and input data obtained by concatenating the reconstructed image with the clinical information into the second neural network to determine the lung volume.

17. The apparatus of claim 13, further comprising
a training unit configured to train the artificial intelligence model including a first neural network and a second neural network using training data through a supervised learning method, wherein
the training unit is further configured to:
input a 2D medical image of the training data to the first neural network for performing image encoding;
predict a lung volume by inputting, to the second neural network, data obtained by concatenating clinical information of the training data with an output value of the first neural network; and
train the first neural network and the second neural network so that a difference between the predicted lung volume and an actual lung volume of the training data is reduced.

18. The apparatus of claim 13, further comprising
a training unit configured to train the artificial intelligence model including a first neural network and a second neural network using training data through a supervised learning method, wherein
the training unit comprises:
a first training unit configured to train the first neural network through a self-supervised learning method by inputting 2D medical image of the training data into the first neural network including an encoding process and a decoding process; and
a second training unit configured to determine feature information on a two-dimensional medical image of the training data through the training-completed first neural network, predict the lung volume by inputting, into the second neural network, data obtained by concatenating the feature information with clinical information of the training data, and train the second neural network to reduce a difference between the predicted lung volume and an actual lung volume of the training data.

19. The apparatus of claim 13, further comprising
a training unit configured to train the artificial intelligence model including a first neural network and a second neural network using training data through a supervised learning method, wherein
the training unit is configured to:
generate a reconstructed image by inputting a 2D medical image of the training data into the first neural network including an encoding process and a decoding process;
predict a lung volume by inputting, into the second neural network, information obtained by concatenating the reconstructed image with clinical information of the training data; and
train the first neural network and the second neural network so that a difference between the predicted lung volume and an actual lung volume of the training data is reduced.

20. A computer-readable recording medium on which a computer program for performing the method of claim 1 is recorded.
